(19) United States
(12) Patent Application Publication
(10) Pub. No.: US 2022/0346708 A1
(43) Pub. Date: Nov. 3, 2022

(54) DEMENTIA DETERMINATION SYSTEM

(71) Applicant: Panasonic Intellectual Property Management Co., Ltd., Osaka (JP)

(72) Inventors: Hirobumi NAKAJIMA, Kyoto (JP); Kengo ABE, Nara (JP); Yosuke AKASHI, Osaka (JP)

(21) Appl. No.: 17/624,210

(22) PCT Filed: Jul. 10, 2020
(86) PCT No.: PCT/JP2020/027117

§ 371 Date: Dec. 30, 2021

(30) Foreign Application Priority Data
Jul. 24, 2019 (JP) 2019-136354

(51) Int. Cl. A61B 5/00; A61B 5/16

(52) U.S. Cl. CPC A61B 5/4842; A61B 5/4088; A61B 5/16

(57) ABSTRACT

A dementia determination system includes: an obtainer that obtains a consecutive press ratio which is a ratio of a total number of consecutive presses of a button of an operating device to a total number of presses of the button of the operating device, the consecutive presses being presses of the button made at time intervals each shorter than or equal to a reference time interval, the operating device being used by a user to operate an electric device; a determiner that determines a dementia level of the user based on the consecutive press ratio obtained by the obtainer; and an outputter that outputs dementia information indicating the dementia level determined by the determiner, wherein the determiner determines that the dementia level is higher as the consecutive press ratio is higher.

### TECHNICAL FIELD

The present invention relates to a dementia determination system and the like for determining a dementia level.

### BACKGROUND ART

In recent years, the proportion of elderly people in the total population has been increasing. With the advent of such an aging society, the problem of dementia is thought to become apparent. Such a problem of dementia is actively taken up in the mass media such as TV programs.

There are various levels of dementia from mild to severe, and if it is known that a person is in the stage of mild cognitive impairment (MCI) before the person becomes dementia, the onset of dementia can be suppressed by rehabilitation training or the like. Patent Literature 1 (PTL 1) discloses a dual task performance ability evaluation method capable of evaluating a subject's ability to perform a dual task. The dual task is one of the rehabilitation menus for dementia and involves using the mind while moving the body.

### CITATION LIST

### Patent Literature

[PTL 1] International Patent Publication No. 2016/148199

### SUMMARY OF INVENTION

### Technical Problem

The present invention provides a dementia determination system and the like that can determine the dementia level based on the history of operations on an electric device.

### Solution to Problem

The dementia determination system according to one aspect of the present invention includes: an obtainer that obtains a consecutive press ratio which is a ratio of a total number of consecutive presses of a button of an operating device to a total number of presses of the button of the operating device, the consecutive presses being presses of the button made at time intervals each shorter than or equal to a reference time interval, the operating device being used by a user to operate an electric device; a determiner that determines a dementia level of the user based on the consecutive press ratio obtained by the obtainer; and an outputter that outputs dementia information indicating the dementia level determined by the determiner, wherein the determiner determines that the dementia level is higher as the consecutive press ratio is higher.

The program according to one aspect of the present invention is a program for causing a computer to execute a dementia determination process, the dementia determination process including: obtaining a consecutive press ratio which is a ratio of a total number of consecutive presses of a button of an operating device to a total number of presses of the button of the operating device, the consecutive presses being presses of the button made at time intervals each shorter than or equal to a reference time interval, the operating device being used by a user to operate an electric device; determining a dementia level of the user based on the consecutive press ratio obtained by the obtaining; and outputting dementia information indicating the dementia level determined by the determining, wherein in the determining, the dementia level is higher as the consecutive press ratio is higher.

### Advantageous Effects of Invention

The dementia determination system or the like according to one aspect of the present invention can determine a dementia level based on the history of operations on an electric device.

### BRIEF DESCRIPTION OF DRAWINGS

FIG. 1 is a conceptual diagram showing a configuration of a dementia determination system according to Embodiment.

FIG. 2 is a block diagram showing a configuration of a dementia determination device according to Embodiment.

FIG. 3 is a time chart showing a timing at which the operating device according to Embodiment transmits a signal.

FIG. 4 is a data configuration diagram showing a signal detection result according to Embodiment.

FIG. 5 is a diagram for explaining a method of specifying a button press for one time.

FIG. 6 is a diagram for explaining a criterion for determining consecutive presses.

FIG. 7 is a diagram for explaining the consecutive press ratio.

FIG. 8 is a diagram showing the correlation between the consecutive press ratio and the score of the forgetfulness consultation program (MSP) for a plurality of users.

FIG. 9 is a flowchart showing an operation of the dementia determination device according to Embodiment.

FIG. 10 is a conceptual diagram showing a configuration of a dementia determination system in a variation.

FIG. 11 is a screen transition diagram showing transition of information displayed on a screen of an electric device in the variation.

FIG. 12 is a screen configuration diagram showing a display area of information displayed on the screen of the electric device in the variation.

FIG. 13 is a flowchart showing the operation of the dementia determination device according to the variation.

### DESCRIPTION OF EMBODIMENTS

Hereinafter, embodiments will be described in details with reference to the drawings. It should be noted that each of the embodiments described below shows a comprehensive or specific example. Numerical values, shapes, materials, components, arrangement positions and connecting forms of components, operation orders, and the like shown in the following embodiments are merely examples, and do not limit the present invention. In addition, among the components in the following embodiments, components not described in the independent claims are described as arbitrary components.

In addition, each of a plurality of drawings used in the following description is a schematic diagram, and does not necessarily show strictly accurate values and the like.

### EMBODIMENT

### [Configuration]

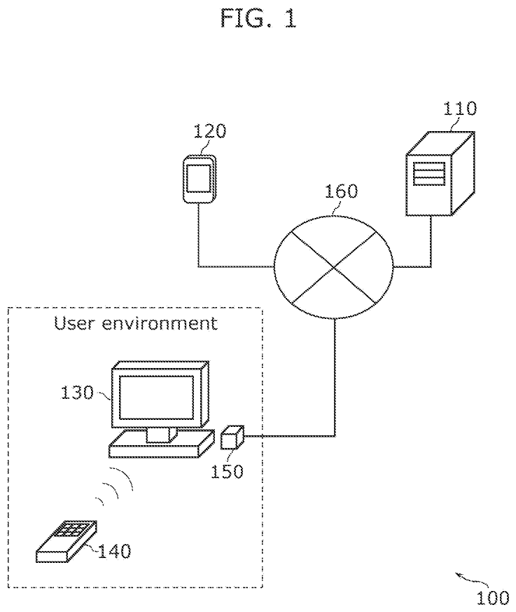

First, the configuration of the dementia determination system in Embodiment will be described. FIG. 1 is a conceptual diagram showing the configuration of the dementia determination system according to Embodiment. Dementia determination system 100 shown in FIG. 1 is a system for determining a dementia level, and includes dementia determination device 110, terminal device 120, electric device 130, operating device 140, sensor device 150, and the like.

Here, the dementia level corresponds to the degree of dementia. The dementia level may be the degree of suspicion of dementia or the possibility of dementia. In addition, a level corresponding to a range of a healthy person, a level corresponding to a range of mild cognitive impairment (MCI: Mild Cognitive Impairment) which is milder than dementia, and the like may be set as dementia levels, respectively.

In addition, the dementia level may be set in two stages: a normal level corresponding to a range of a healthy person and an abnormal level corresponding to a range between mild cognitive impairment and dementia. Then, the abnormal level may be further subdivided into a plurality of sub-levels, such as mild cognitive impairment, mild dementia, moderate dementia, and severe dementia.

Dementia determination device 110 is a device for determining a dementia level. That is, the dementia determination in dementia determination system 100 is substantially performed in dementia determination device 110. For example, dementia determination device 110 is a computer that performs information processing. Dementia determination device 110 may be a single device or may include a plurality of auxiliary devices. In addition, dementia determination device 110 may be installed in a user environment, or may be installed in a position different from the user environment.

In the example of FIG. 1, dementia determination device 110 is installed at a position different from the user environment. Then, dementia determination device 110 is connected via communication network 160 to sensor device 150 installed in a user environment. Then, dementia determination device 110 obtains a detection result from sensor device 150.

In addition, dementia determination device 110 is connected to terminal device 120 via communication network 160. For example, dementia determination device 110 outputs dementia information by transmitting dementia information indicating a dementia level to terminal device 120.

Terminal device 120 is a device that receives the dementia information transmitted from dementia determination device 110. For example, terminal device 120 is a computer that communicates information, and is specifically a tablet terminal, a smart phone, a mobile phone, a personal computer, or the like. Terminal device 120 may be fixedly installed or may be portable.

In addition, terminal device 120 may be installed in a user environment, or may be installed in a position different from the user environment. For example, terminal device 120 is used by a caregiver or the like of a user in a user environment. Terminal device 120 may be used by a user in a user environment.

Electric device 130 is a device operated by a user using operating device 140. For example, electric device 130 is a household electric appliance, and is basically a television receiver. A television receiver is also simply called a television. Electric device 130 may be an audio device, an air conditioner, a lighting device, a refrigerator, a rice cooker, a multifunctional telephone, or the like. Electric device 130 may be an electric toilet seat. In addition, electric device 130 may be a device having a screen. In the example of FIG. 1, electric device 130 is a television. In addition, electric device 130 is basically installed in a user environment.

Operating device 140 is a device for operating electric device 130. For example, operating device 140 is a remote controller for electric device 130 and includes buttons for operating electric device 130. Operating device 140 may be a smart phone or the like for operating electric device 130. Then, buttons for operating electric device 130 may be displayed on the screen included in operating device 140 by a GUI. In addition, operating device 140 may be an operation panel integrated with electric device 130.

Specifically, in the example of FIG. 1, operating device 140 is a remote controller for electric device 130 which is a television. Then, operating device 140 transmits a signal using infrared rays. Accordingly, electric device 130 which is a television is operated. In addition, operating device 140 is basically used in a user environment. It should be noted that when electric device 130 is an electric toilet seat, operating device 140 is, for example, a remote controller for remotely controlling electric device 130, which is an electric toilet seat, with a finger push button.

Sensor device 150 is a device that detects a signal transmitted from operating device 140. For example, sensor device 150 is an infrared sensor. Sensor device 150 is basically installed near electric device 130. Accordingly, sensor device 150 can appropriately detect a signal transmitted from operating device 140. In addition, for example, sensor device 150 transmits a detection result to dementia determination device 110. Sensor device 150 may transmit a detection result to dementia determination device 110 each time a signal is detected.

Alternatively, sensor device 150 may record and store the detection result in an internal memory. Then, when receiving the transmission request of the detection result from dementia determination device 110, sensor device 150 may transmit the detection result recorded and stored in the internal memory.

Communication network 160 is a network for dementia determination device 110, terminal device 120, sensor device 150, and the like to communicate. For example, communication network 160 is the Internet. Communication network 160 may be a LAN (local area network) or the like.

It should be noted that dementia determination device 110 may be dementia determination system 100. In other words, dementia determination system 100 may include the components of dementia determination device 110, and may not include the other components. Then, the other components may be included in other systems. In addition, the communication between the components may be wired communication or wireless communication. Then, communication network 160 may be a wired network or a wireless network.

In addition, although FIG. 1 shows one user environment, a plurality of user environments corresponding to a plurality of users may exist. Then, components corresponding to electric device 130, operating device 140, and sensor device 150 may be included in each user environment. Then, dementia determination device 110 may determine the dementia level of each user.

In addition, for example, one user environment may correspond to one room for the elderly in an apartment complex for the elderly. Then, terminal device 120 may be installed in the management room of the apartment complex for the elderly. Dementia determination device 110 may be installed in the management room of the apartment complex for the elderly, or may be installed outside because it determines a dementia level of each user in a plurality of apartment complexes for the elderly.

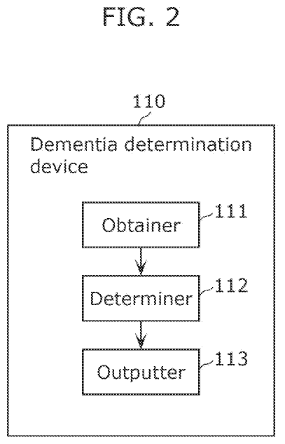

FIG. 2 is a block diagram showing the configuration of dementia determination device 110 shown in FIG. 1. Dementia determination device 110 includes obtainer 111, determiner 112, and outputter 113.

Obtainer 111 is a processing unit that obtains information, and specifically obtains information for determining a dementia level. Obtainer 111 may be a general-purpose or dedicated electric circuit.

For example, obtainer 111 may obtain information by receiving information through wired or wireless communication. In this case, obtainer 111 may include a terminal or an antenna for communication. Specifically, obtainer 111 may obtain the detection result of the signal transmitted from operating device 140 as information by receiving the detection result of the signal transmitted from operating device 140 as information from sensor device 150.

In addition, for example, obtainer 111 may receive a detection result of a signal transmitted from operating device 140 from sensor device 150 and obtain information for determining a dementia level from the detection result.

In addition, for example, obtainer 111 may obtain information via an input interface such as a mouse, a keyboard, or a touch panel. In addition, obtainer 111 may include such an input interface. In addition, obtainer 111 may obtain information from a memory such as a recording medium. In addition, obtainer 111 may include a connection interface for connecting to a recording medium or the like. In addition, obtainer 111 may perform information processing on the obtained information and obtain a result of the information processing as new information.

More specifically, obtainer 111 obtains a consecutive press ratio with respect to operating device 140 as information for determining a dementia level. The consecutive press ratio is a ratio of the number of consecutive presses to the number of presses. The number of presses is the number of times a button of operating device 140 is pressed. The number of consecutive presses is the number of times the button of operating device 140 is pressed at time intervals each shorter than a predetermined reference time interval. The reference time interval is, for example, 5 seconds, but may be another time interval. The reference time interval is only needed to be determined empirically or experimentally as appropriate. The reference time interval may be a fixed time or a dynamically determined time. It should be noted that the details of consecutive presses will be described later.

In addition, for example, obtainer 111 may obtain the number of presses or the number of consecutive presses. Specifically, obtainer 111 may obtain the number of presses and the number of consecutive presses from the detection result obtained from sensor device 150. Then, obtainer 111 may obtain the consecutive press ratio by calculating the consecutive press ratio from the number of presses and the number of consecutive presses.

Determiner 112 is a processing unit that determines a dementia level, and specifically determines a dementia level according to the information obtained by obtainer 111. Determiner 112 may be a general-purpose or dedicated electric circuit.

Specifically, determiner 112 determines the dementia level based on the consecutive press ratio obtained by obtainer 111. In particular, determiner 112 determines that the dementia level is relatively higher as the consecutive press ratio is higher. For example, when the consecutive press ratio is the first ratio, determiner 112 determines the dementia level to be the first level, and when the consecutive press ratio is the second ratio higher than the first ratio, determiner 112 determines the dementia level to be the second level that is higher than the first level.

In addition, determiner 112 does not always need to determine that the dementia level is higher as the consecutive press ratio is higher. According to other conditions and the like, determiner 112 does not need to exceptionally determine that the dementia level is higher as the consecutive press ratio is higher. In addition, for example, when the consecutive press ratio is within a predetermined range, determiner 112 may determine that the dementia level is the same level.

In addition, for example, when the consecutive press ratio is higher than or equal to a predetermined threshold, determiner 112 may determine the dementia level to be a normal level corresponding to the range of a healthy person. Then, when the consecutive press ratio is higher than the predetermined threshold, determiner 112 may determine the dementia level to be an abnormal level corresponding to the range between mild cognitive impairment and dementia.

In addition, determiner 112 may use other parameters for determining the dementia level, without being limited to the consecutive press ratio. That is, determiner 112 may determine the dementia level based on the consecutive press ratio and other parameters.

The other parameter is, for example, the number of presses obtained by obtainer 111. For example, determiner 112 determines that the dementia level is higher as the number of presses is smaller. Determiner 112 may determine that the dementia level is higher as the consecutive press ratio is higher and the number of presses is smaller, based on the evaluation value obtained by weighting and adding the consecutive press ratio and the number of presses.

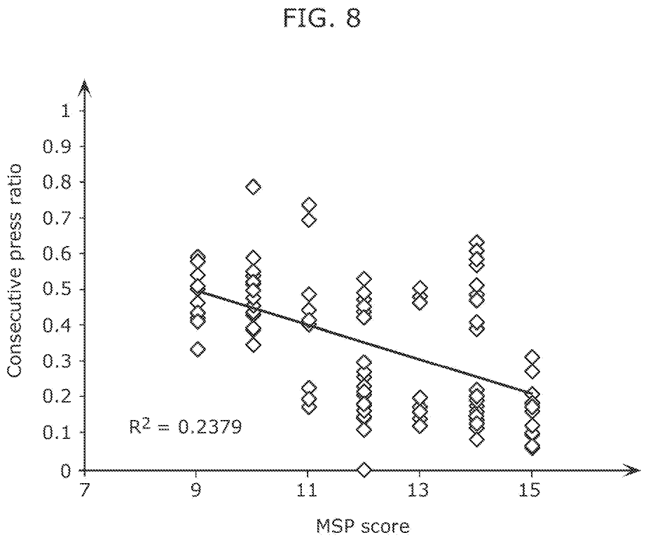

It should be noted that the relationship between the dementia level (healthy level) and the number of presses, and the relationship between the dementia level (healthy level) and the consecutive press ratio will be described later with reference to FIG. 8.

Output unit 113 is a processing unit that outputs information, and specifically outputs dementia information indicating the dementia level determined by determiner 112, and the like. Output unit 113 may be a general-purpose or dedicated electric circuit.

For example, outputter 113 may output information by transmitting information by wire or wireless communication. In this case, outputter 113 may include a terminal or an antenna for communication. Then, outputter 113 may output dementia information by transmitting dementia information to terminal device 120.

In addition, for example, outputter 113 may output information via an output interface such as a display, a speaker, or a touch panel. Specifically, outputter 113 may output the information by displaying the information on a display, or may output the information as audio from a speaker. In addition, outputter 113 may include such an output interface.

In addition, outputter 113 may output information to a memory such as a recording medium. In addition, outputter 113 may include a connection interface for connecting to a recording medium or the like.

In addition, the configuration of the terminal, antenna, input interface, connection interface, or the like in obtainer 111 and the configuration of the terminal, antenna, input interface, connection interface, or the like in outputter 113 may be partially or wholly shared.

### [Knowledge of the Inventors]

When a healthy user changes to the MCI state, various disorders such as executive function disorder or disorientation appear as symptoms. When the cognitive function decreases, the ability of the conscious part is greatly reduced, and in order to grasp the tendency of the user to change to the state of MCI, it is necessary to detect the deterioration of the user's judgment such as disorientation.

As such, the inventors paid attention to the consecutive presses of a button of operating device 140. In general, when a user is watching TV for no purpose, does not know what the user is doing, and just presses a button on operating device 140, or the like, consecutive presses of the button on operating device 140 occur. The inventors have found a configuration that detects a decrease in cognitive function mainly due to disorientation by continuously pressing a button on operating device 140. Hereinafter, the dementia level determination operations of dementia determination system 100 based on such consecutive presses of a button of operation device 140 will be described.

### [Method of Specifying the Consecutive Press Ratio]

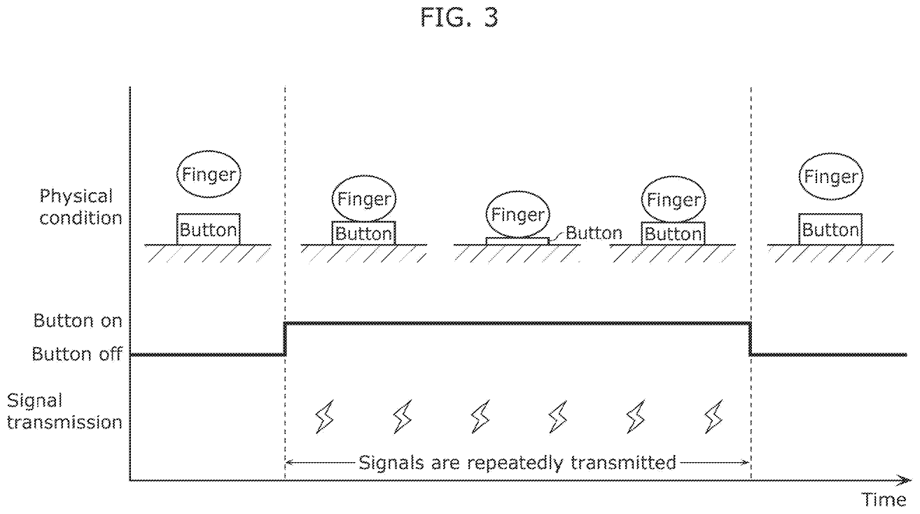

First, a method of specifying the consecutive press ratio based on the signals transmitted from operating device 140 by sensor device 150 will be described. FIG. 3 is a time chart showing the timing at which operating device 140 shown in FIG. 1 transmits a signal.

While the button of operating device 140 is being pressed by the user's finger, the button is in an on state, and the signal is repeatedly transmitted. In particular, the button is in an on state from the time the button starts to be pressed until the time when the button returns. Therefore, the signal is repeatedly transmitted from the time when the button starts to be pressed until the time when the button returns. For example, this signal is transmitted by infrared rays approximately every 200 ms when the button is in an on state. Therefore, if the time period from the time when the button starts to be pressed until the time when the button returns is 1 to 2 seconds, the signal is transmitted 5 to 10 times.

Electric device 130 receives the signal transmitted from operating device 140 and operates according to the received signal. For example, at that time, electric device 130 stops receiving the signal during a few seconds after receiving the signal. Alternatively, electric device 130 discards the signal received during the few seconds. Accordingly, electric device 130 suppresses a double operation.

Sensor device 150 also receives a signal transmitted from operating device 140, similarly to electric device 130. That is, sensor device 150 detects a signal transmitted from operating device 140. However, even after detecting a signal, sensor device 150 detects the signal without stopping the detection. Then, sensor device 150 does not discard the signal detected later, but also includes information indicating the signal detected later in the detection result.

Accordingly, information indicating a signal transmitted a plurality of times for one press is included in the detection result.

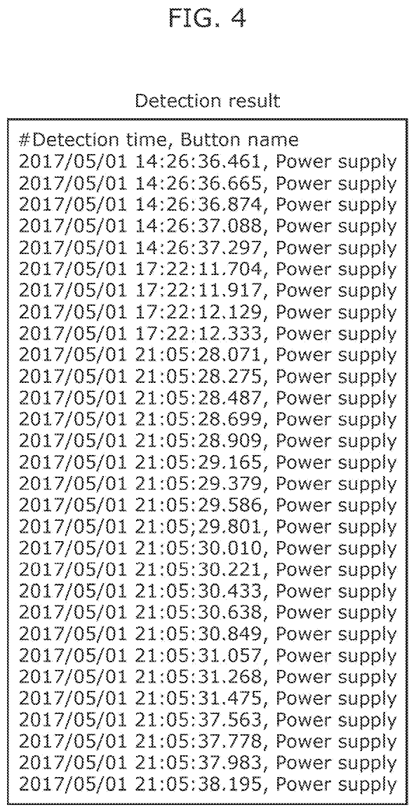

FIG. 4 is a data configuration diagram showing a detection result of a signal for dementia determining device 110 shown in FIG. 2 and the like to determine a dementia level. The detection result shown on the left side of FIG. 4 is an example of the detection result obtained by sensor device 150.

For example, the detection result includes the detection time and the button name. The detection time is a time when sensor device 150 detects a signal, and is a time when sensor device 150 receives a signal transmitted from operating device 140. The button name is a name of the button identified by the detected signal as the pressed button.

It should be noted that in the example of FIG. 4, the detection result of a signal transmitted when the power button is pressed is shown so as not to complicate the description. The detection result obtained by sensor device 150 may include a detection result of a signal transmitted when another button is pressed. In addition, the detection result may be expressed in another format. For example, a button identifier may be used instead of a button name.

Sensor device 150 detects a signal transmitted from operating device 140 in about 200 milliseconds. Therefore, the original detection result is a detection result in 200 milliseconds. Then, the detection result in 200 milliseconds is transmitted from sensor device 150 to dementia determination device 110. Obtainer 111 of dementia determination device 110 obtains the detection result by receiving the detection result transmitted from sensor device 150.

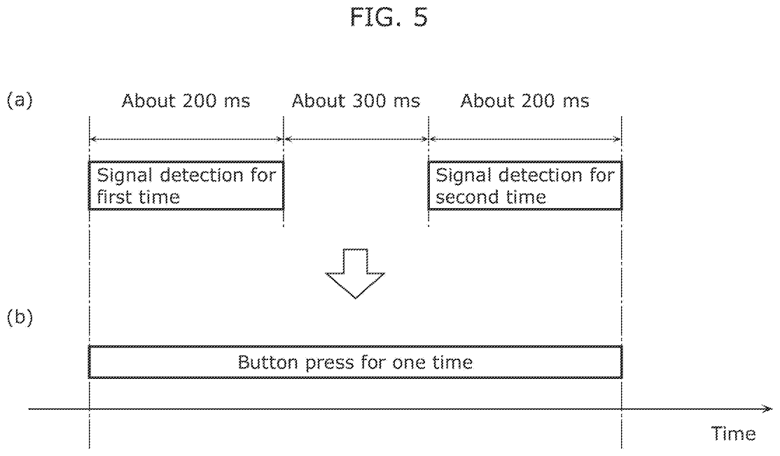

Then, obtainer 111 specifies a button press for one time by aggregating the detection results in units of 200 milliseconds. FIG. 5 is a diagram for explaining a method of specifying a button press for one time. As shown in (a) in FIG. 5, the signal may be detected a plurality of times in one second, but it is difficult for the user whose dementia level is determined to press the button a plurality of times in one second.

As such, for example, signal detections for a plurality of times with a time interval of 1 second or less are regarded as a button press for one time. When the time interval between the signal detections for two times (each for 200 milliseconds) shown in (a) in FIG. 5 is 300 milliseconds, which is not more than 1 second, the signal detection for two times is regarded as a button press for one time of 700 ms in duration.

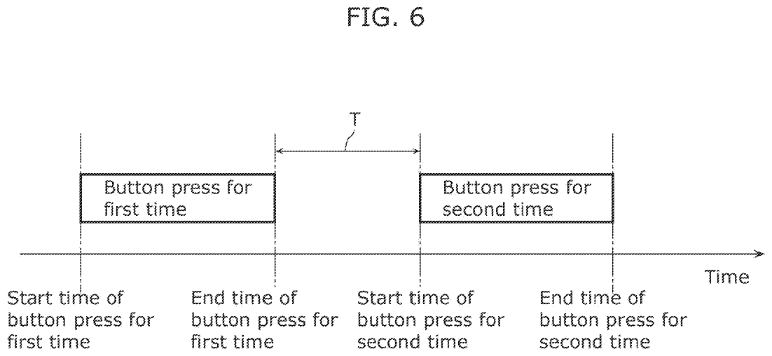

In this way, after the button press for one time is specified, obtainer 111 specifies the number of consecutive presses. FIG. 6 is a diagram for explaining a criterion for determining consecutive presses.

As shown in FIG. 6, when the button press for the second time is performed after the button press for the first time, the time interval from the end time of the button press for the first time to the start time of the button press for the second time is assumed to be T. When time interval T is shorter than or equal to the reference time interval, it is determined that the button press for the first time and the button press for the second time are consecutive presses. That is, FIG. 6 shows two consecutive presses. It should be noted that, in FIG. 6, the case where time interval T is shorter than or equal to the reference time interval, the consecutive presses may be determined to be a single continuous press. The reference time interval is, for example, 5 seconds, but may be another time. The reference time interval is only needed to be determined empirically or experimentally as appropriate. The reference time interval may be a fixed time or a dynamically determined time.

It should be noted that in the determination of consecutive presses, it does not matter whether the button pressed for the first time and the button pressed for the second time are the same. In other words, the button pressed for the first time and the button pressed for the second time may be the same button or different buttons.

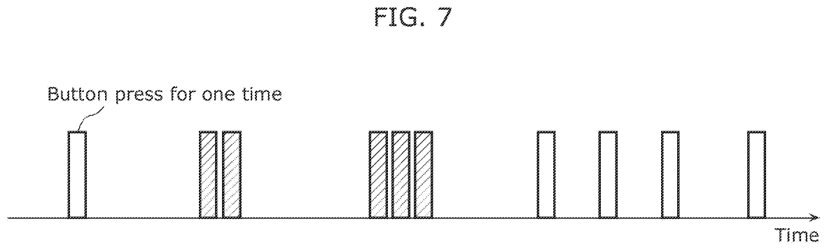

Then, obtainer 111 obtains the ratio of the number of consecutive presses of the button to the number of presses of the button as the consecutive press ratio. FIG. 7 is a diagram for explaining the consecutive press ratio.

In FIG. 7, one bar graph shows a button press for one time, and the hatched bar graphs show button presses determined to be consecutive presses. As shown in FIG. 7, for example, when the button presses for 10 times are specified in a target period (for example, 1 day) and it is determined that 5 times of them are consecutive presses, 5/10=1/2 is obtained as the consecutive press ratio.

It should be noted that the format of the detection result, the method of obtaining the number of presses, the method of obtaining the number of consecutive presses, and the method of obtaining the consecutive press ratio described above are merely examples, and are not limited to the above examples.

The consecutive press ratio specified in this way has a correlation with the cognitive function of the user. FIG. 8 is a diagram showing the correlation between the consecutive press ratio and the score of the forgetfulness consultation program (MSP) for a plurality of users. It should be noted that the forgetfulness consultation program is a screening test program for finding Alzheimer's Disease (AD) patients. It should be noted that, one point plotted in FIG. 8 shows the average value of the continuous push ratio of one user (for example, the average value for one month of the consecutive press ratio for one day as a target period).

As shown in FIG. 8, correlation coefficient R between the consecutive press ratio described above and the MSP score is R=−0.488 (R²=0.2379). In addition, the p value in this case is p=0.000, which is extremely lower than the significance level of 0.05. That is, it can be said that the consecutive press ratio and the MSP score have a clear correlation. It should be noted that the consecutive press ratio indicates that the cognitive function decreases as the numerical value increases, whereas the MSP score indicates that the cognitive function decreases as the numerical value decreases, so that both have a negative correlation.

It should be noted that in FIG. 8, the correlation coefficient is calculated for users with the MSP score of 9 or more, but even if the correlation coefficient is calculated including users with moderate AD having a low MSP score and users with severe AD, there is a clear correlation between the consecutive press ratio and the MSP score. That is, the determination of the cognitive function using the consecutive press ratio can distinguish users ranging from severe AD users to healthy users. However, since the correlation is stronger when the correlation coefficient is calculated only for users with a high MSP score, it can be said that the determination of cognitive function using the consecutive press ratio is more suitable for distinguishing mild AD users, MCI users, and healthy users.

### [Method of Specifying the Consecutive Press Ratio]

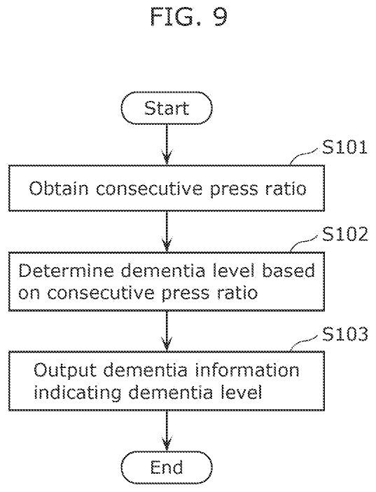

The operation of dementia determination device 110 using the consecutive press ratio as described above will be described. FIG. 9 is a flowchart showing the operation of dementia determination device 110 shown in FIG. 2 and the like. Dementia determination device 110 performs the operation shown in FIG. 9 in determining the dementia level of the user.

First, obtainer 111 of dementia determination device 110 obtains a consecutive press ratio (S101). The consecutive press ratio is a ratio of a number of consecutive presses of a button of operating device 140 to a number of presses of the button of operating device 140. The consecutive presses are presses of the button made at time intervals each shorter than or equal to a reference time interval. The operating device is used by a user to operate electric device 130. For example, obtainer 111 may obtain a detection result from sensor device 150 that detects a signal transmitted from operating device 140, and may obtain a consecutive press ratio from the detection result.

Next, determiner 112 of dementia determination device 110 determines the dementia level of the user based on the consecutive press ratio obtained by obtainer 111 (S102). At that time, determiner 112 determines that the dementia level is higher as the consecutive press ratio is higher. For example, when the consecutive press ratio is higher than or equal to a threshold, determiner 112 may determine the dementia level to be an abnormal level corresponding to the range between mild cognitive impairment and dementia. Then, when the consecutive press ratio is lower than the threshold, determiner 112 may determine the dementia level to be a normal level corresponding to the range between mild cognitive impairment and dementia.

Then, outputter 113 of dementia determination device 110 outputs dementia information indicating the dementia level determined by determiner 112 (S103). For example, outputter 113 may output dementia information by transmitting the dementia information to terminal device 120.

Accordingly, dementia determination device 110 can determine the dementia level according to the daily work performed by the user. In addition, the consecutive press ratio for determining the dementia level is set irrespective of whether the operation is correct or erroneous, and is hardly influenced by user's preference. Then, it is presumed that the consecutive press ratio decreases as the cognitive ability increases. Therefore, dementia determination device 110 can appropriately determine the dementia level based on the consecutive press ratio.

### (Variation)

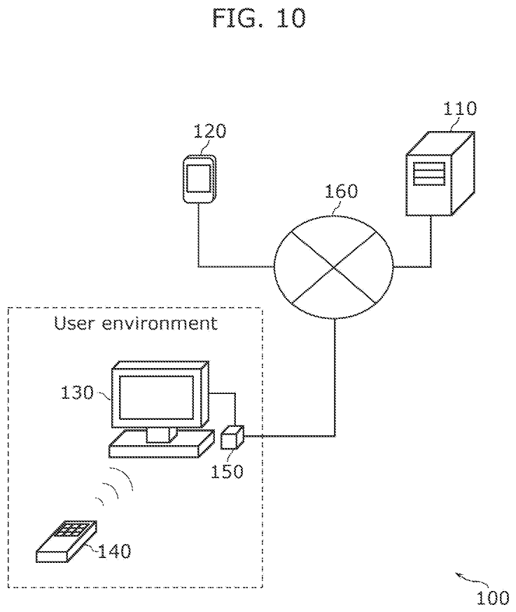

FIG. 10 is a conceptual diagram showing a varied configuration of dementia determination system 100 shown in FIG. 1. In this variation, sensor device 150 is connected to electric device 130 and controls the operation of electric device 130 by communicating with electric device 130. That is, sensor device 150 operates as a control device that controls the operation of electric device 130.

For example, outputter 113 of dementia determination device 110 may display the dementia information on the screen of electric device 130 via sensor device 150 by outputting the dementia information. Specifically, outputter 113 of dementia determination device 110 outputs dementia information to sensor device 150. Then, sensor device 150 receives the dementia information from dementia determination device 110, and transmits the dementia information to electric device 130. Then, electric device 130 receives the dementia information from sensor device 150 and displays the dementia information on the screen.

Accordingly, dementia determination device 110 can notify the user of electric device 130 of the dementia level.

In addition, for example, by outputting training content for improving the cognitive ability of the user, outputter 113 of dementia determination device 110 may display the training content on the screen of electric device 130 via sensor device 150.

Specifically, similarly to the example of the dementia information, outputter 113 of dementia determination device 110 outputs the training content to sensor device 150 based on the dementia level. Then, sensor device 150 receives the training content from dementia determination device 110, and transmits the training content to electric device 130. Then, electric device 130 receives the training content from sensor device 150 and displays the dementia information on the screen.

Accordingly, dementia determination device 110 can prompt the user of electric device 130 to perform training for improving cognitive ability. The training content displayed on the screen of electric device 130 is training content for an operation in which operating device 140 is used. For example, by performing an operation on electric device 130 using operating device 140 according to the training content, training for improving cognitive ability is performed. Since such an operation involves the movement of the user's finger, it is effective in improving cognitive ability.

In addition, training content may be set for each dementia level. Accordingly, training for improving the user's cognitive ability is performed according to the training content set for the dementia level of the user. The training content may be recorded in a memory inside or outside dementia determination device 110 in advance.

In addition, when the dementia level of the user is determined to be a level corresponding to mild cognitive impairment or dementia, outputter 113 of dementia determination device 110 may cause the training content to be displayed on the screen of electric device 130. That is, when the determined dementia level is higher than the predetermined reference level, outputter 113 of dementia determination device 110 may cause the training content to be displayed on the screen of electric device 130. Accordingly, dementia determination device 110 can suppress the training content from being displayed when the training is not necessary.

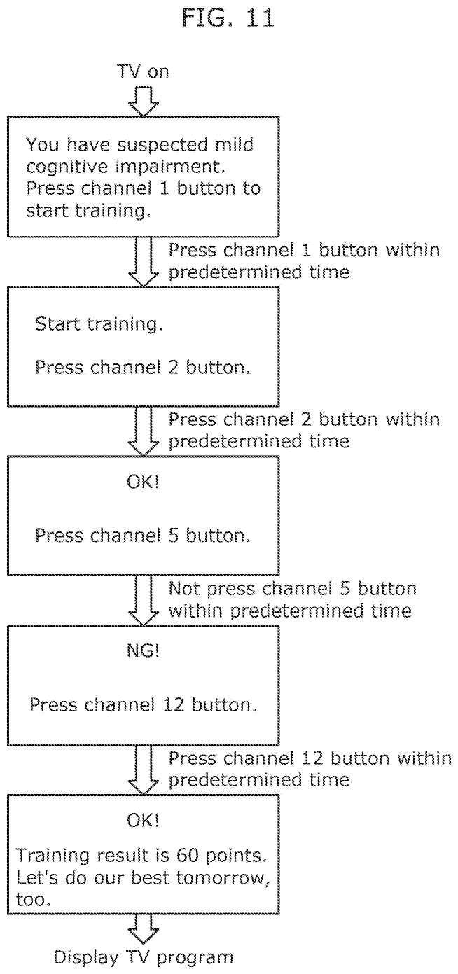

FIG. 11 is a screen transition diagram showing transition of information displayed on the screen of electric device 130 shown in FIG. 10. For example, dementia information and training content are displayed as shown in FIG. 11.

In the example of FIG. 11, when the television, which is electric device 130, is turned on, a notification that there is a suspicion of mild cognitive impairment is displayed on the screen of the television. This notification is displayed when the television is first turned on every day, and may not be displayed after the second time. In addition, this notification corresponds to dementia information indicating a dementia level determined based on a past operation. For example, this notification may indicate a dementia level determined based on the operations during the past day, the past week, the past month or the like.

Then, when the training is started, the user is prompted to press the channel 1 button. Here, the training is started when the channel 1 button is pressed within a predetermined time. That is, the training content is displayed.

For example, when the channel 1 button is pressed within a predetermined time, next, the start of training is notified, and the user is prompted to press the channel 2 button. When the channel 2 button is pressed within a predetermined time, a notification of OK is given, and then the user is prompted to press the channel 5 button. When the channel 5 button has not been pressed within a predetermined time, a notification of NG is given, and then the user is prompted to press the channel 12 button. When the channel 12 button is pressed within a predetermined time, the notification of OK is given.

Then, the training result is displayed by the score. At that time, the score may be set according to the number of times the operation is appropriately performed according to the instruction indicated by the training content. In addition, the consecutive press ratio during training may be reflected in the score. Thereafter, the display of the television program is started as usual.

It should be noted that dementia determination device 110 may reflect the consecutive press ratio during training in the determination of the dementia level. In addition, dementia determination device 110 may newly determine the dementia level based on the consecutive press ratio in the determination target period including the training period, and may display dementia information indicating the newly determined dementia level on the screen after training.

In addition, the predetermined time for pressing the channel button may be set according to the dementia level of the user. In addition, the press of the channel button is repeated three times in the training in the above description, but the number of repetitions may be set based on the dementia level of the user.

For example, a plurality of training contents having different predetermined times for button pressing and different numbers of repetitions may be set for a plurality of dementia levels in advance. Then, the training may be performed according to the training content set for the dementia level of the user among the plurality of training content set for the plurality of dementia levels in advance.

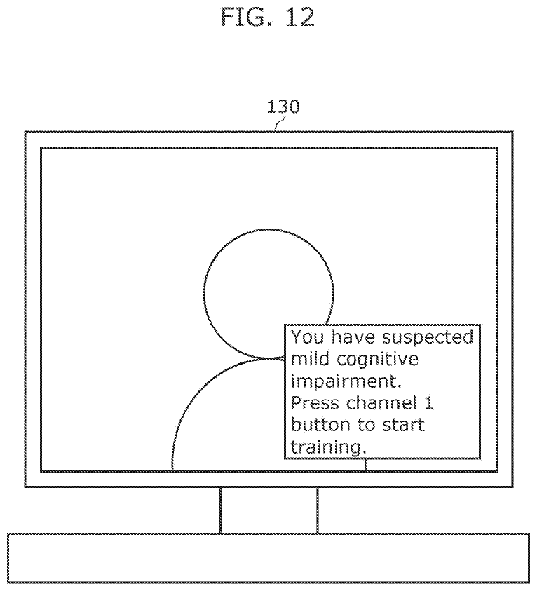

FIG. 12 is a screen configuration diagram showing a display area of information displayed on the screen of electric device 130 shown in FIG. 10. The dementia information and the training content may be displayed in a partial area of the screen instead of the entire screen. In the example of FIG. 12, dementia information and training content are displayed in the lower right area of the screen. Then, the television program is displayed in another area of the screen.

It should be noted that a button that is rarely used may be used as a button used for training and a button used for starting training. Accordingly, it is possible to suppress the operation for viewing the television program from being hindered.

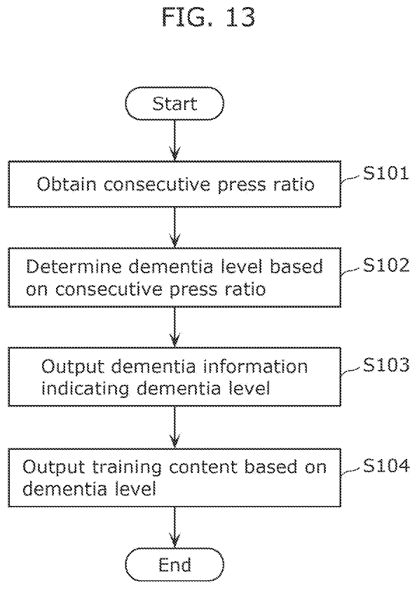

FIG. 13 is a flowchart showing the operation of dementia determination system 100 shown in FIG. 10 and the like. Dementia determination device 110 according to the variation performs the operation shown in FIG. 13 in determining the dementia level of the user.

First, obtainer 111 of dementia determination device 110 obtains a consecutive press ratio (S101). Next, determiner 112 of dementia determination device 110 determines the dementia level of the user based on the consecutive press ratio obtained by obtainer 111 (S102). Then, outputter 113 of dementia determination device 110 outputs dementia information indicating the dementia level determined by determiner 112 (S103).

These processes are basically the same as in the example of FIG. 9. In the present variation, outputter 113 of dementia determination device 110 may display the dementia information on the screen of electric device 130 by outputting the dementia information.

In addition, in the present variation, outputter 113 of dementia determination device 110 outputs the training content on the screen of electric device 130 by outputting the training content based on the dementia level determined by determiner 112 (S104). For example, outputter 113 of dementia determination device 110 may display the training content set for the dementia level on the screen of electric device 130 by outputting the training content set for the dementia level determined by determiner 112.

Accordingly, dementia determination device 110 can notify the user of the determination result of the dementia level via electric device 130 used for the determination of the dementia level. Then thereby, dementia determination device 110 can prompt the user to improve the life rhythm and the like. In addition, dementia determination device 110 can prompt the user to improve cognitive ability by displaying the training content on the screen of electric device 130.

In addition, for example, the operation on electric device 130 is performed using operating device 140 according to the training content. Dementia determination device 110 may reflect the operation performed according to the training content in the next determination.

Specifically, in the next determination, obtainer 111 of dementia determination device 110 newly obtains the consecutive press ratio in the determination target period including the training period in which the training content is displayed. Then, determiner 112 of dementia determination device 110 newly determines the dementia level based on the consecutive press ratio newly obtained by obtainer 111.

Accordingly, dementia determination device 110 can appropriately determine the dementia level improved by the training.

It should be noted that in Embodiment and the variation, sensor device 150 may be incorporated in electric device 130. Then, electric device 130 may have a role of sensor device 150. Furthermore, dementia determination device 110 may be incorporated in electric device 130, or a plurality of components included in dementia determination device 110 may be incorporated in electric device 130. Then, electric device 130 may have a role of dementia determination device 110.

Dementia determination system 100 may authenticate each user from among a plurality of users in the user environment and determine the dementia level of the authenticated user from among the plurality of users. For example, a plurality of operating devices may be assigned to a plurality of users, respectively. Then, the user may be authenticated according to the operating device used for the operation among the plurality of operating devices. Alternatively, one operating device may authenticate each user by fingerprint authentication.

In addition, when determining the dementia level based on the consecutive press ratio, dementia determination system 100 may compare the consecutive press ratio in the past state where the user was healthy with the consecutive press ratio in the current state. Then, when the consecutive press ratio in the current state is higher than the consecutive press ratio in the past state, dementia determination system 100 may determine that the dementia level in the current state is higher than the dementia level in the past state.

That is, the threshold for the consecutive press ratio for determining the dementia level of the user in the current state may be set based on the consecutive press ratio in the past state in which the user was healthy. Alternatively, in order to set such a threshold, instead of the consecutive press ratio in a past state where the user was healthy, an average consecutive press ratio in a healthy state of a plurality of users may be used.

In addition, when determining the dementia level based on the consecutive press ratio, dementia determination system 100 may use other determination criteria in addition to the consecutive press ratio. That is, dementia determination system 100 may determine the dementia level based on the consecutive press ratio and other parameters.

As described above, dementia determination system 100 according to one aspect of the present invention has been described according to Embodiment and the like, but the present invention is not limited to Embodiment and the like. The present invention includes a form obtained by applying variations that can be conceived by those skilled in the art to Embodiment and the like, and another form realized by arbitrarily combining a plurality of components in Embodiment and the like.

For example, a process performed by a specific component may be performed by another component. In addition, the order in which the processes are performed may be changed, or a plurality of processes may be performed in parallel.

In addition, the present invention can be implemented not only as dementia determination system 100 but also as a dementia determination method including steps performed by each component included in dementia determination system 100. For example, those steps are executed by a computer system including a processor, a memory, an input/output circuit, and the like. Then, the present invention can be realized as a program for causing a computer system to execute the steps included in those methods. It should be noted that a computer system may be simply referred to as a computer.

In addition, the present invention can be realized as a non-transitory computer-readable recording medium on which the above-described program is recorded. The recording medium may be an optical disk such as a CD-ROM, a magnetic disk such as a hard disk drive, a magneto-optical disk (MO), a semiconductor memory such as a flash memory, or other non-transitory computer-readable recording medium. In addition, the program may be recorded in a recording medium in advance, or may be recorded in the recording medium by being supplied to the recording medium via a communication network.

For example, when the present invention is implemented by a program, each step is executed by executing the program using hardware resources such as a processor, a memory, and an input/output circuit of a computer system. That is, each step is executed by the processor obtaining data from a memory or an input/output circuit or the like and performing an operation, or outputting the operation result to the memory or the input/output circuit or the like. Any type of processor can be used as the processor for executing the program.

In addition, each of the plurality of components included in dementia determination system 100 and the like may be realized as a dedicated or general-purpose circuit. These components may be realized as one circuit or as a plurality of circuits.

In addition, the plurality of components included in dementia determination system 100 and the like may be realized as an LSI (Large Scale Integration) that is an integrated circuit (IC). These components may be individually integrated into one chip, or may be integrated into one chip so as to include some or all of the components. These components may be provided on one or more chips of one device, or may be provided on a plurality of chips of a plurality of devices.

In addition, the LSI may be called a system LSI, a super LSI, or an ultra LSI depending on the degree of integration. In addition, the integrated circuit may be realized by a dedicated circuit or a general-purpose processor. A programmable FPGA (Field Programmable Gate Array) or a reconfigurable processor in which connection and setting of internal circuit cells can be reconfigured may be used.

Furthermore, if an integrated circuit technology that replaces the LSI appears due to the advancement of the semiconductor technology or another derivative technology, naturally, the integrated circuit of a plurality of components included in dementia determination system 100 may be integrated using the technology.

Finally, a plurality of aspects of dementia determination system 100 and the like will be described as examples. These aspects may be appropriately combined. In addition, any of the features and the like described in Embodiments 1, 2 and the like may be added.

### (Aspect 1)

Dementia determination system 100 according to one aspect of the present invention includes obtainer 111, determiner 112, and outputter 113.

Obtainer 111 obtains a consecutive press ratio which is a ratio of a number of consecutive presses of a button of operating device 140 to a number of presses of the button of operating device 140. The consecutive presses are presses of the button made at time intervals each shorter than or equal to a reference time interval. The operating device is used by a user to operate electric device 130. Determiner 112 determines a dementia level of the user based on the consecutive press ratio obtained by obtainer 111. At that time, determiner 112 determines that the dementia level is higher as the consecutive press ratio is higher. Output unit 113 outputs dementia information indicating the dementia level determined by determiner 112.

Accordingly, dementia determination system 100 can determine the dementia level according to the daily work performed by the user. In addition, the consecutive press ratio for determining the dementia level is set irrespective of whether the operation is correct or erroneous, and is hardly influenced by user's preference. Then, it is presumed that the consecutive press ratio increases as the cognitive ability decreases. Therefore, dementia determination system 100 can appropriately determine the dementia level based on the consecutive press ratio.

### (Aspect 2)

For example, when the consecutive press ratio is higher than or equal to the threshold, determiner 112 may determine that the dementia level is an abnormal level corresponding to a range between mild cognitive impairment and dementia. Then, when the consecutive press ratio is lower than the threshold, determiner 112 may determine that the dementia level is a normal level corresponding to a range of a healthy person. Accordingly, dementia determination system 100 can appropriately determine whether the dementia level of the user is a normal level or an abnormal level.

### (Aspect 3)

For example, outputter 113 may cause the dementia information to be displayed on a screen of electric device 130 by outputting the dementia information. Accordingly, dementia determination system 100 can notify the user of the dementia level of the user. Therefore, dementia determination system 100 can prompt the user to improve the life rhythm.

### (Aspect 4)

For example, by outputting training content for an operation in which operating device 140 is used based on the dementia level determined by determiner 112, outputter 113 may cause the training content to be displayed on a screen of electric device 113. Accordingly, dementia determination system 100 can prompt the user for training based on the dementia level determined.

### (Aspect 5)

For example, when the dementia level determined by determiner 112 is higher than a reference level, outputter 113 may cause the training content to be displayed on the screen by outputting the training content. Accordingly, dementia determination system 100 can prompt the user for training when the user is determined to have mild cognitive impairment or dementia and the like.

### (Aspect 6)

For example, outputter 113 may cause the training content to be displayed on the screen of electric device 130 by outputting the training content set for the dementia level determined by determiner 112. Accordingly, dementia determination system 100 can prompt the user for training suitable for the dementia level determined.

### (Aspect 7)

For example, obtainer 111 may newly obtain the consecutive press ratio in the determination target period including a training period in which the training content is displayed. Determiner 112 may newly determine the dementia level based on the consecutive press ratio newly obtained by obtainer 111. Accordingly, dementia determination system 100 can collect more information and determine the dementia level. In addition, dementia determination system 100 can determine a dementia level improved by training.

### (Aspect 8)

The program according to one aspect of the present invention is a program for causing a computer to execute a dementia determination process including obtaining step (S101), determining step (S102), and outputting step (S103).

In obtaining step (S101), a consecutive press ratio is obtained which is a ratio of a number of consecutive presses of a button of operating device 140 to a number of presses of the button of operating device 140. The consecutive presses are presses of the button made at time intervals each shorter than or equal to a reference time interval. The operating device is used by a user to operate electric device 130.

In determination step (S102), a dementia level of the user based on the consecutive press ratio obtained in obtaining step (101) is determined. In addition, it is determined in determination step (S102) that the dementia level is higher as the consecutive press ratio is higher. In output step (S103), dementia information indicating the dementia level determined in determination step (S102) is output.

Accordingly, the program can cause the computer to execute an appropriate determination of the dementia level.

### REFERENCE SIGNS LIST

100 Dementia determination system
111 Obtainer
112 Determiner
113 Outputter
130 Electric device
140 Operating device

1. A dementia determination system comprising:
an obtainer that obtains a consecutive press ratio which is a ratio of a total number of consecutive presses of a button of an operating device to a total number of presses of the button of the operating device, the consecutive presses being presses of the button made at time intervals each shorter than or equal to a reference time interval, the operating device being used by a user to operate an electric device;
a determiner that determines a dementia level of the user based on the consecutive press ratio obtained by the obtainer; and
an outputter that outputs dementia information indicating the dementia level determined by the determiner,
wherein the determiner determines that the dementia level is higher as the consecutive press ratio is higher.

2. The dementia determination system according to claim 1,
wherein when the consecutive press ratio is higher than or equal to a threshold, the determiner determines that the dementia level is an abnormal level corresponding to a range between mild cognitive impairment and dementia, and
when the consecutive press ratio is lower than the threshold, the determiner determines that the dementia level is a normal level corresponding to a range of a healthy person.

3. The dementia determination system according to claim 1,
wherein the outputter causes the dementia information to be displayed on a screen of the electric device by outputting the dementia information.

4. The dementia determination system according to claim 1,
wherein by outputting training content for an operation in which the operating device is used based on the dementia level determined by the determiner, the outputter causes the training content to be displayed on a screen of the electric device.

5. The dementia determination system according to claim 4,
wherein when the dementia level determined by the determiner is higher than a reference level, the outputter causes the training content to be displayed on the screen by outputting the training content.

6. The dementia determination system according to claim 4,
wherein the outputter causes the training content to be displayed on the screen by outputting the training content set for the dementia level determined by the determiner.

7. The dementia determination system according to claim 4,
wherein the obtainer newly obtains the consecutive press ratio in a determination target period including a training period in which the training content is displayed, and
the determiner newly determines the dementia level based on the consecutive press ratio newly obtained by the obtainer.

8. The dementia determination system according to claim 1,
wherein when determining the total number of the consecutive presses, the button of the operating device which is pressed comprises a single button or a plurality of buttons.

9. A computer-readable non-transitory recording medium having recorded thereon a program for causing a computer to execute a dementia determination process,
the dementia determination process including:
obtaining a consecutive press ratio which is a ratio of a total number of consecutive presses of a button of an operating device to a total number of presses of the button of the operating device, the consecutive presses being presses of the button made at time intervals each shorter than or equal to a reference time interval, the operating device being used by a user to operate an electric device;
determining a dementia level of the user based on the consecutive press ratio obtained in the obtaining; and
outputting dementia information indicating the dementia level determined in the determining,
wherein in the determining, the dementia level is determined to be higher as the consecutive press ratio is higher.
